(19) United States
(12) Patent Application Publication
(10) Pub. No.: US 2026/0232634 A1
(43) Pub. Date: Aug. 13, 2026

(54) TREATMENT OF PEDIATRIC CANCERS USING GYLCOGEN SYNTHASE KINASE FORM B INHIBITORS

(71) Applicant: Actuate Therapeutics Inc., Fort Worth, TX (US)

(72) Inventor: Andrew MAZAR, Fort Worth, TX (US)

(21) Appl. No.: 19/471,492

(22) PCT Filed: Apr. 3, 2024
(86) PCT No.: PCT/US2024/022760

§ 371 Date: Oct. 1, 2025

Related U.S. Application Data
(60) Provisional application No. 63/493,909, filed on Apr. 3, 2023.
(60) Provisional application No. 63/494,858, filed on Apr. 7, 2023.

(51) Int. Cl. A61K 31/407; A61K 31/4745; A61K 31/495; A61K 31/664; A61K 45/06; A61P 35/00

(52) U.S. Cl. CPC A61K 31/407; A61K 31/4745; A61K 31/495; A61K 31/664; A61K 45/06; A61P 35/00

(57) ABSTRACT

Methods of treating cancer in a pediatric patient in need thereof, comprising administering to said patient 9-ING-41 in combination with an additional therapeutic agent, are provided.

### CROSS-REFERENCE TO RELATED APPLICATIONS

The application claims the benefit of U.S. Provisional Patent Application No. 63/493,909, filed Apr. 3, 2023, and U.S. Provisional Patent Application No. 63/494,858, filed Apr. 7, 2023. The entirety of each aforementioned application is incorporated by reference herein in its entirety.

### TECHNICAL FIELD

The disclosure pertains to methods of treating cancer in pediatric patients.

### BACKGROUND

GSK-3 is a serine/threonine kinase initially described as a key regulator of metabolism, specifically glycogen biosynthesis. It has a role in diverse disease processes including cancer, immune disorders, metabolic disorders, and neurological disorders through modulation of many substrates. GSK-3 has two ubiquitously expressed and highly conserved isoforms, GSK-3α and GSK-3β.

GSK-3β is particularly important in tumor progression and modulation of oncogenes (including beta-catenin, cyclin D1 and c-Myc), cell cycle regulators (e.g. p27^{Kip1}) and mediators of epithelial-mesenchymal transition (e.g. zinc finger protein SNAI1, Snail). Aberrant overexpression of GSK-3β has been shown to promote tumor growth and chemotherapy resistance in various solid tumors including colon, ovarian, and pancreatic cancers and glioblastoma through differential effects on the pro-survival nuclear factor kappa-light-chain-enhancer of activated B cells (NF-κB) and c-Myc pathways as well on tumor necrosis factor-related apoptosis-inducing ligand (TRAIL) and p53-mediated apoptotic mechanisms.

3-(5-Fluorobenzofuran-3-yl)-4-(5-methyl-5H-[1,3]dioxolo[4,5-f]indol-7-yl) pyrrole-2,5-dione (“9-ING-41” or “elraglusib”) is a GSK-3β inhibitor that has the following chemical structure:

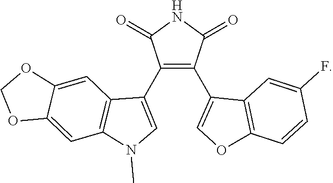 [SMILES: Cn1cc(C2=C(c3coc4ccc(F)cc34)C(=O)NC2=O)c2cc3c(cc21)OCO3]

9-ING-41 is a first-in-class, intravenously (IV) administered, maleimide-based small molecule potent selective GSK-3β inhibitor with significant pre-clinical and clinical antitumor activity that involves G0-G1 and G2-M phase arrest.

The synthesis, properties, and/or biological activity of 9-ING-41 are set forth in U.S. Pat. No. 8,207,216; Gaisina et al., From a Natural Product Lead to the Identification of Potent and Selective Benzofuran-3-yl-(indol-3-yl) maleimides as Glycogen Synthase Kinase 3β Inhibitors That Suppress Proliferation and Survival of Pancreatic Cancer Cells, J. Med. Chem. 2009, 52, 1853-1863; and Hilliard, et al., Glycogen synthase kinase 3β inhibitors induce apoptosis in ovarian cancer cells and inhibit in-vivo tumor growth, Anti-Cancer Drugs 2011, 22:978-985. 9-ING-41 has been reported to be useful for the treatment of certain cancers, including brain, lung, breast, ovarian, bladder, neuroblastoma, renal, and pancreatic cancers, as well as for treatment of traumatic brain injury.

### SUMMARY

The present disclosure provides methods of treating cancer in a pediatric patient in need thereof, comprising administering to said patient 9-ING-41 in combination with an additional therapeutic agent.

In some aspects, the cancer is an alveolar rhabdomyosarcoma, embryonal CNS tumor NOS, neuroblastoma, osteosarcoma, progressive ependymoma, Ewing's sarcoma, rhabdomyosarcoma, pediatric glioblastoma multiforme (GBM), diffuse interstitial pontine glioma (DIPG)/diffuse midline glioma (DMG), Wilm's tumor, recurrent ependymoma, CIC rearranged sarcoma, high grade astrocytoma, adrenocortical carcinoma, embryonal CNS, ependymoma, glioma, astrocytoma, or adrenocortical.

In some aspects, the cancer is refractory or recurrent cancer.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

As used in the specification including the appended claims, the singular forms “a,” “an,” and “the” include the plural, and reference to a particular numerical value includes at least that particular value, unless the context clearly dictates otherwise.

When a range of values is expressed, an exemplary embodiment includes from the one particular value and/or to the other particular value. All ranges are inclusive and combinable. Further, reference to values stated in ranges includes each and every value within that range. When values are expressed as approximations, by use of the preposition “about,” it will be understood that the particular value forms another embodiment. The term “about” as used herein when referring to a measurable value such as an amount, a temporal duration, and the like, is meant to encompass reasonable variations of the value, such as, for example, ±10% from the specified value. For example, the phrase “about 5-20 mg/kg” can include ±10% of 5, ±10% of 20 or from 4.5 to 22, inclusive of 5-20.

It is to be appreciated that certain features of the disclosure which are, for clarity, described herein in the context of separate embodiments, may also be provided in combination in a single embodiment. Conversely, various features of the disclosure that are, for brevity, described in the context of a single embodiment, may also be provided separately or in any subcombination.

As used herein, and the term “pediatric patient(s)”, refers to a human patient who is 23 years old or younger.

As used herein, whether by itself or in conjunction with another term or terms, it should be understood that the phrases “method of treating” and “method of treatment” may be used interchangeably with the phrase “for use in the treatment of” a particular disease.

As used herein, whether by themselves or in conjunction with another term or terms, “treats,” “treating,” “treated,” and “treatment,” refer to and include ameliorative, palliative, and/or curative uses and results, or any combination thereof.

As used herein, whether used alone or in conjunction with another term or term, “therapeutic agent” refers to a compound or composition that (a) treats a particular condition, symptom, disorder, or disease described herein; (b) attenuates, ameliorates, or eliminates one or more symptoms of a particular condition, disorder, or disease described herein; (c) delays the onset or relapse (reoccurrence) of a particular condition, symptom, disorder, or disease described herein; (d) prevents the onset of a particular condition, symptom, disorder, or disease described herein. It should be understood that the terms “therapeutic” and “therapeutically effective” encompass any one of the aforementioned effects (a)-(d), either alone or in combination with any of the others (a)-(d).

The term “administering” means either directly administering a compound or composition of the present invention, or administering a prodrug, derivative or analog which will form an equivalent amount of the active compound or substance within the body.

As used herein, the term “refractory cancer” refers to a cancer that is resistant to previous chemotherapeutic treatments. Refractory cancers include cancers that have exhibited resistance at the beginning of previous chemotherapeutic treatment(s), or that have become resistant during the course of previous chemotherapeutic treatment(s).

As used herein, the term “recurrent cancer” refers to a cancer that has recurred (come back), usually after a period of time during which the cancer could not be detected. The cancer may come back to the same place as the original (primary) tumor or to another place in the body.

As used herein, the term “CIC-rearranged sarcoma” refers to a class of small round-cell tumors that fall under the Ewing sarcoma group of cancers. Although historically grouped with Ewing sarcomas, these tumors are genetically distinct and tend to be more aggressively metastatic than Ewing sarcomas on average.

### Methods of Use

In some aspects, the disclosure provides methods of treating cancer in a pediatric patient in need thereof, comprising administering to the patient 9-ING-41 in combination with an additional therapeutic agent.

In some embodiments of the disclosed methods, the cancer is an alveolar rhabdomyosarcoma, embryonal CNS tumor NOS, neuroblastoma, osteosarcoma, progressive ependymoma, Ewing's sarcoma, rhabdomyosarcoma, pediatric glioblastoma multiforme (GBM), diffuse interstitial pontine glioma (DIPG)/diffuse midline glioma (DMG), Wilm's tumor, recurrent ependymoma, CIC rearranged sarcoma, high grade astrocytoma, adrenocortical carcinoma, embryonal CNS cancer, ependymoma, glioma, astrocytoma, or adrenocortical cancer.

In some embodiments of the disclosed methods, the cancer is a sarcoma.

In some embodiments of the disclosed methods, the sarcoma is an alveolar rhabdomyosarcoma, osteosarcoma, Ewing sarcoma, rhabdomyosarcoma, or CIC rearranged sarcoma.

In some embodiments of the disclosed methods, the sarcoma is alveolar rhabdomyosarcoma.

In some embodiments of the disclosed methods, the sarcoma is rhabdomyosarcoma.

In some embodiments of the disclosed methods, the sarcoma is osteosarcoma.

In some embodiments of the disclosed methods, the sarcoma is Ewing sarcoma.

In some embodiments of the disclosed methods, the sarcoma is CIC rearranged sarcoma.

In some embodiments of the disclosed methods, the cancer is an embryonal CNS tumor NOS (not otherwise specified).

In some embodiments of the disclosed methods, the cancer is a neuroblastoma.

In some embodiments of the disclosed methods, the cancer is a progressive ependymoma.

In some embodiments of the disclosed methods, the cancer is a pediatric glioblastoma multiforme (GBM).

In some embodiments of the disclosed methods, the cancer is a diffuse interstitial pontine glioma (DIPG)/diffuse midline glioma (DMG).

In some embodiments of the disclosed methods, the cancer is a Wilm's tumor.

In some embodiments of the disclosed methods, the cancer is a recurrent ependymoma.

In some embodiments of the disclosed methods, the cancer is a high grade astrocytoma.

In some embodiments of the disclosed methods, the cancer is an adrenocortical carcinoma.

In some embodiments of the disclosed methods, the cancer is an embryonal CNS cancer.

In some embodiments of the disclosed methods, the cancer is an ependymoma.

In some embodiments of the disclosed methods, the cancer is a glioma.

In some embodiments of the disclosed methods, the cancer is an astrocytoma.

In some embodiments of the disclosed methods, the cancer is a adrenocortical cancer.

In some embodiments of the disclosed methods, the cancer is a refractory or recurrent cancer.

In some embodiments of the disclosed methods, the cancer is a refractory cancer.

In other embodiments of the disclosed methods, the cancer is a recurrent cancer.

In some embodiments of the disclosed methods, the cancer is both refractory and recurrent.

In some aspects, the methods of the disclosure are performed on a pediatric patient.

In some embodiments of the disclosed methods, the pediatric patient is between 0-23 years old, such as for example, one of: 1 month old, 2 months old, 3 months old, 4 months old, 5 months old, 6 months old, 7 months old, 8 months old, 9 months old, 10 months old, 11 months old, 12 months old, 1 year old, 2 years old, 3 years old, 4 years old, 5 years old, 6 years old, 7 years old, 8 years old, 9 years old, 10 years old, 11 years old, 12 years old, 13 years old, 14 years old, 15 years old, 16 years old, 17 years old, 18 years old, 19 years old, 20 years old, 21 years old, 22 years old, or 23 years old.

In some embodiments of the disclosed methods, the pediatric patient is between 0-18 years old, such as for example, one of: 1 month old, 2 months old, 3 months old, 4 months old, 5 months old, 6 months old, 7 months old, 8 months old, 9 months old, 10 months old, 11 months old, 12 months old, 1 year old, 2 years old, 3 years old, 4 years old, 5 years old, 6 years old, 7 years old, 8 years old, 9 years old, 10 years old, 11 years old, 12 years old, 13 years old, 14 years old, 15 years old, 16 years old, 17 years old, or 18 years old.

In some embodiments of the disclosed methods, the pediatric patient is between 0-14 years old, such as for example, one of: 1 month old, 2 months old, 3 months old, 4 months old, 5 months old, 6 months old, 7 months old, 8 months old, 9 months old, 10 months old, 11 months old, 12 months old, 1 year old, 2 years old, 3 years old, 4 years old, 5 years old, 6 years old, 7 years old, 8 years old, 9 years old, 10 years old, 11 years old, 12 years old, 13 years old, or 14 years old.

In some embodiments of the disclosed methods, the 9-ING-41 is administered in an amount of about 5-20 mg/kg of patient weight, for example, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg.

In some embodiments of the disclosed methods, the 9-ING-41 is administered intravenously.

In some embodiments of the disclosed methods, the 9-ING-41 is administered in an amount of about 7 mg/kg.

In some embodiments of the disclosed methods, the 9-ING-41 is administered in an amount of about 9.3 mg/kg.

In some embodiments of the disclosed methods, the 9-ING-41 is administered in an amount of about 12.4 mg/kg.

In some embodiments of the disclosed methods, the 9-ING-41 is administered in an amount of about 15 mg/kg.

In some embodiments of the disclosed methods, the 9-ING-41 is administered at a frequency of once per week over a 21-day cycle.

In some embodiments of the disclosed methods, the 9-ING-41 is administered at a frequency of twice per week over a 21-day cycle.

In some embodiments of the disclosed methods, the 9-ING-41 is administered at a frequency of three times per week over a 21-day cycle.

In some embodiments of the disclosed methods, the 9-ING-41 is administered at a frequency of four times per week over a 21-day cycle.

In some embodiments of the disclosed methods, the 9-ING-41 is administered at a frequency of five times per week over a 21-day cycle.

In some embodiments of the disclosed methods, the 9-ING-41 is administered at a frequency of six times per week over a 21-day cycle.

In some embodiments of the disclosed methods, the 9-ING-41 is administered at a frequency of seven times per week over a 21-day cycle.

In some embodiments of the disclosed methods, the 9-ING-41 is administered in an amount of about 7 mg/kg administered at a frequency of twice per week over a 21-day cycle.

In some embodiments of the disclosed methods, the 9-ING-41 is administered in an amount of about 9.3 mg/kg administered at a frequency of twice per week over a 21-day cycle.

In some embodiments of the disclosed methods, the 9-ING-41 is administered in an amount of about 12.4 mg/kg administered at a frequency of twice per week over a 21-day cycle.

In some embodiments of the disclosed methods, the 9-ING-41 is administered in an amount of about 15 mg/kg administered at a frequency of twice per week over a 21-day cycle.

In some embodiments of the disclosed methods, the additional therapeutic agent comprises irinotecan, cyclophosphamide, topotecan, temozolomide, a PI3 kinase, protein kinase B (AKT) inhibitor, or a mTOR inhibitor.

In some embodiments of the disclosed methods, the additional therapeutic agent comprises irinotecan.

In some embodiments of the disclosed methods, the irinotecan is administered in an amount of 20-50 mg/m2/day, such as, for example, one of: 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/m2/day.

In some embodiments of the disclosed methods, the irinotecan is administered intravenously.

In some embodiments of the disclosed methods, the irinotecan is administered on days 1-5 of every 21 day cycle.

In some embodiments of the disclosed methods, the irinotecan is administered intravenously in an amount of 50 mg/m2/day.

In some embodiments of the disclosed methods, the irinotecan is administered intravenously in an amount of 50 mg/m2/day on days 1-5 of every 21 day cycle.

In some embodiments of the disclosed methods, the irinotecan is administered intravenously over 90 minutes in an amount of 50 mg/m2/day on days 1-5 of every 21 day cycle.

In some embodiments of the disclosed methods, the additional therapeutic agent comprises temozolomide.

In some embodiments of the disclosed methods, the temozolomide is administered orally.

In some embodiments of the disclosed methods, the temozolomide is administered on days 1-5 of every 21 day cycle.

In some embodiments of the disclosed methods, the temozolomide is administered orally in an amount of 100 mg/m2/dose to patients having a body surface area at least 0.5 m².

In some embodiments of the disclosed methods, the temozolomide is administered orally in an amount of 2.5 mg/kg to patients having a body surface area less than 0.5 m².

In some embodiments of the disclosed methods, the temozolomide is administered orally in an amount of 100 mg/m2/dose on days 1-5 of every 21 day cycle.

In some embodiments of the disclosed methods, the additional therapeutic agent comprises both irinotecan and temozolomide, wherein each of irinotecan and temozolomide is administered as disclosed herein.

In some embodiments of the disclosed methods, the additional therapeutic agent comprises cyclophosphamide.

In some embodiments of the disclosed methods, the cyclophosphamide is administered intravenously.

In some embodiments of the disclosed methods, the cyclophosphamide is administered on days 1-5 of every 21 day cycle.

In some embodiments of the disclosed methods, the cyclophosphamide is administered intravenously in an amount of 400 mg/m2/dose on days 1-5 of every 21 day cycle.

In some embodiments of the disclosed methods, the cyclophosphamide is administered intravenously over 30 minutes in an amount of 400 mg/m2/dose on days 1-5 of every 21 day cycle.

In some embodiments of the disclosed methods, the additional therapeutic agent comprises topotecan.

In some embodiments of the disclosed methods, the topotecan is administered intravenously.

In some embodiments of the disclosed methods the topotecan is administered on Days 1 through 5 of every 21 day cycle.

In some embodiments of the disclosed methods the topotecan is administered intravenously over 30 minutes in an amount of 1.2 mg/m2/dose on Days 1 through 5 of every 21 day cycle.

In some embodiments of the disclosed methods the topotecan is administered intravenously over 30 minutes in an amount of 1.2 mg/m2/dose once on Days 1 through 5.

In some embodiments of the disclosed methods, the additional therapeutic agent comprises both cyclophosphamide and topotecan, wherein each of cyclophosphamide and topotecan is administered as disclosed herein.

In some embodiments of the disclosed methods, the additional therapeutic agent comprises a PI3K inhibitor, such as, for example, one or more of copanlisib, idelalisib, umbralisib, duvelisib, alpelisib, inavolisib, gedatolisib, or paxalisib.

In some embodiments of the disclosed methods, the additional therapeutic agent comprises a protein kinase B (AKT) inhibitor, such as, for example, capivasertib, miransertib, or ipatasertib.

In some embodiments of the disclosed methods, the additional therapeutic agent comprises a mTor inhibitor, such as, for example, sirolimus, everolimus, and temsirolimus.

In some embodiments of the disclosed methods, the additional therapeutic agent comprises a PD-L1 inhibitor, such as, for example, atezolizumab (Tecentriq), avelumab (Bavencio), or durvalumab (Imfinzi).

In some embodiments of the disclosed methods, the additional therapeutic agent comprises a PD-1 inhibitor, such as, for example, Pembrolizumab (Keytruda), Nivolumab (Opdivo), or Cemiplimab (Libtayo).

In some embodiments of the disclosed methods, the additional therapeutic agent comprises a CTLA-4 inhibitor, such as, for example, Ipilimumab (Yervoy) and tremelimumab (Imjuno).

In some embodiments, the methods of the disclosure result in a complete response according to RECIST 1.1 criteria.

In some embodiments, the methods of the disclosure result in a partial response according to RECIST 1.1 criteria.

In some embodiments, the methods of the disclosure result in stable disease according to RECIST 1.1 criteria.

In some embodiments, the methods of the disclosure increase patient overall survival (OS). As used herein, the term “overall survival” is defined as the time from initiation of the therapy to death from any cause.

In some embodiments, the methods of the disclosure increase patient progression-free survival (PFS). As used herein, the term “progression-free survival” is defined as the time from initiation of the therapy to until objective tumor progression or death.

It will be understood that in the methods of the disclosure, the 9-ING-41 may be administered in a pharmaceutical composition comprising the 9-ING-41 and at least one pharmaceutically acceptable excipient.

Similarly, the additional therapeutic agent used in the methods of the disclosure may be administered in a pharmaceutical composition comprising the therapeutic agent and at least one pharmaceutically acceptable excipient.

It should be understood that references herein to methods of treating cancer using the disclosed compounds or compositions should also be interpreted as references to: (i) the disclosed compounds or compositions for use in methods of treating cancer; and/or (ii) the use of the disclosed compounds or compositions in the manufacture of a medicament for treating cancer.

### EXAMPLES

### Example 1. Human Pharmacokinetics of 9-ING-41 from Study

### NCT03678883:

| Dose | 9.3 mg/kg (current dose) |
|---|---|
| Cₘₐₓ, ng/ml | 6,414 ± 3,550 |
| T_{1/2}, h | 18.9 ± 6.3 |
| AUC₍₀₋₂₄₎, ng · h/mL | 42,043 ± 12,437 |
| AUC_{(0-inf)}, ng · h/mL | 45,734 ± 14,139 |

### Example 2—Clinical Study

| Investigational | 9-ING-41 | | |
|---|---|---|---|
| Product | | | |
| Title | Phase 1/2 Study of 9-ING-41, a Glycogen Synthase Kinase 3 Beta (GSK-3β) | | |
| | Inhibitor, as a Single Agent or with Irinotecan, Temozolomide/Irinotecan or | | |
| | Cyclophosphamide/Topotecan in Pediatric Patients with Refractory Malignancies | | |
| No. of Patients | Approximately 100 evaluable | | |
| Primary | Phase 1: | | |
| Objectives | To determine the maximum tolerated dose (MTD) and/or recommended | | |
| | Phase 2 dose (RP2D) of 9-ING-41 as a single agent and in combination with | | |
| | Irinotecan in pediatric patients with refractory malignancies. | | |
| | To describe the toxicity profile and DLT of 9-ING-41 as a single agent and | | |
| | in combination with irinotecan, temozolomide/irinotecan, and | | |
| | cyclophosphamide/topotecan in pediatric patients with refractory | | |
| | malignancies. | | |
| | Phase 2 (neuroblastoma-specific arms): | | |
| | To evaluate the efficacy of 9-ING-41 in combination with | | |
| | temozolomide/irinotecan and cyclophosphamide/topotecan in refractory | | |
| | neuroblastoma patients, as defined by response rates, duration of responses, | | |
| | and progression-free survival (PFS). | | |
| | To characterize the safety profile of 9-ING-41 in combination with | | |
| | temozolomide/irinotecan and cyclophosphamide/topotecan in pediatric | | |
| | patients with refractory neuroblastoma. | | |
| Secondary | To explore the efficacy of 9-ING-41 as a single agent and in combination | | |
| Objectives | with irinotecan in this patient population as defined by response rates, | | |
| | duration of responses, and PFS. | | |
| | To characterize the pharmacokinetics of 9-ING-41 in combination with | | |
| | irinotecan, temozolomide/irinotecan, and cyclophosphamide/topotecan, | | |
| | when administered to pediatric patients with refractory malignancies. | | |
| Design | Open label, non-randomized, multi-center, Phase 1/2 | | |
| Endpoints | Safety, via (CTCAE v5.0), will be monitored during the period starting on | | |
| | initiation of study therapy and ending 30 days after the final administration of | | |
| | 9-ING-41. All patients who receive any dose (any amount) of 9-ING-41 will be | | |
| | included in the summaries and listings of safety data. Overall safety profile and | | |
| | tolerability will be characterized by type, frequency, severity, timing, duration, | | |
| | and relationship of study drug of adverse events and laboratory abnormalities. | | |
| | The efficacy endpoints are the following: | | |
| | Objective response rate (ORR), defined as the percent of patients with | | |
| | complete response (CR), partial response (PR) according to RECIST 1.1 | | |
| | criteria or other standard malignancy-specific response criteria, relative | | |
| | to the efficacy population, such as INRC for neuroblastoma. | | |
| | Duration of response (DoR), defined as the time from documentation of | | |
| | tumor response to disease progression. | | |
| | Progression-free survival (PFS), defined as the time from study | | |
| | enrolment until objective tumor progression or death. | | |
| | Overall survival (OS), defined as the time from study entry to death from any | | |
| | cause. | | |
| Population | Inclusion Criteria: | | |
| | Patients must meet ALL the following criteria to be eligible for this study: | | |
| | 1. Age ≤22 years | | |
| | 2. Diagnosis of recurrent or refractory malignancy with histologic verification | | |
| | of malignancy at original diagnosis or relapse, except patients with extra- | | |
| | cranial germ-cell tumors who have elevations of serum tumor markers | | |
| | including alpha-fetoprotein or beta-HCG, and/or patients with intrinsic brain | | |
| | stem tumors or patients with CNS-germ cell tumors and elevations of CSF | | |
| | or serum tumor markers including alpha-fetoprotein or beta-HCG (Applies | | |
| | to Phase 1 only; for Phase 2 arm, see Inclusion Criterion #4). | | |
| | 3. Have either measurable or evaluable disease. Evaluable disease is defined as | | |
| | an assessment of tumor that cannot be measured using a ruler or calipers but | | |
| | can be used to determine disease progression or response, e.g., positive on | | |
| | MIBG scan (with or without bone marrow histology) or bone scan; | | |
| | metastatic bone marrow disease; elevated tumor markers; or presence of a | | |
| | malignant pleural effusion. (Applies to Phase 1 only; for Phase 2 arm, see | | |
| | Inclusion Criterion #4). | | |
| | 4. For the neuroblastoma-specific Phase 2 arm: | | |
| | Patients must have had histologic verification of neuroblastoma or | | |
| | ganglioneuroblastoma or demonstration of neuroblastoma cells in the bone | | |
| | marrow with elevated urinary catecholamines (i.e., >2 × ULN), at the time | | |
| | of initial diagnosis. | | |
| | Documentation of disease (Patients must have at least ONE of the following | | |
| | at the time of enrollment): | | |
| | a. Measurable tumor on MRI or CT scan. Measurable is defined as ≥10 | | |
| | mm in at least one dimension on spiral/helical CT that is MIBG avid or | | |
| | demonstrates increased FDG uptake on PET scan. | | |
| | b. MIBG-avid lesion detected on MIBG scan with positive uptake at a | | |
| | minimum of two sites. This site must represent disease recurrence after | | |
| | completion of therapy, progressive disease on therapy, or refractory | | |
| | disease during induction. If 1-2 MIBG avid sites, then biopsy required at | | |
| | any time of at least one site that verifies neuroblastoma or | | |
| | ganglioneuroblastoma. | | |
| | c. Patients with resistant/refractory soft tissue disease that is not MIBG avid | | |
| | or does not demonstrate increased FDG uptake on PET scan can | | |
| | undergo biopsy to document the presence of viable neuroblastoma. | | |
| | d. Patients with elevated catecholamines (i.e. >2 × ULN) only are NOT | | |
| | eligible for this part of the study. | | |
| | 5. Have current disease state for which there is no known curative therapy or | | |
| | therapy proven to prolong survival with an acceptable quality of life. | | |
| | 6. Have Performance Level: Karnofsky ≥50% for patients >16 years of age | | |
| | and Lansky ≥50 for patients ≤16 years of age | | |
| | 7. Neurologic deficits in patients with CNS tumors must have been relatively | | |
| | stable for at least 7 days prior to study enrollment. Patients with CNS | | |
| | tumors who are receiving steroids must be on a stable or decreasing dose for | | |
| | at least 7 days prior to study entry. Patients who are unable to walk because | | |
| | of paralysis, but who are up in a wheelchair, will be considered ambulatory | | |
| | for the purpose of assessing the performance score. | | |
| | 8. Have fully recovered from the acute clinically significant toxic effects of | | |
| | prior anti-cancer therapy: | | |
| | Myelosuppressive chemotherapy: On first day of treatment be at least | | |
| | 7 days after the last dose of myelosuppressive chemotherapy for single | | |
| | agent 9-ING-41, and at least 14 days after the last dose of | | |
| | myelosuppressive chemotherapy for 9-ING-41 plus chemotherapy | | |
| | combination arms | | |
| | Hematopoietic growth factors: At least 14 days after the last dose of a | | |
| | long-acting growth factor or 7 days for short-acting growth factor | | |
| | Biologic (anti-neoplastic agent): At least 7 days after the last dose of a | | |
| | biologic agent or retinoid | | |
| | Monoclonal antibodies: At least 28 days after the last dose of a | | |
| | monoclonal antibody | | |
| | Radiotherapy: At least 14 days after local palliative radiotherapy | | |
| | (small port); at least 100 days must have elapsed if prior TBI, | | |
| | craniospinal radiotherapy or if ≥50% radiation of pelvis; at least 42 | | |
| | days must have elapsed if other substantial BM radiation | | |
| | Neuroblastoma-specific arms: No interim time prior to study entry | | |
| | is required following prior radiotherapy for non-target lesions. | | |
| | However, patients must not have received radiation for a minimum | | |
| | of 14 days prior to study entry at the site of any lesion that will be | | |
| | identified as a target lesion to measure tumor response. Lesions | | |
| | that have been previously radiated cannot be used as target lesions | | |
| | unless there is radiographic evidence of progression at the site | | |
| | following radiation or a biopsy done following radiation shows | | |
| | viable neuroblastoma. Palliative radiation is allowed to sites that | | |
| | will not be used to measure response during this study. | | |
| | Stem cell transplants (SCT): No evidence of active graft versus host | | |
| | disease and at least 42 days must have elapsed after transplant or stem | | |
| | cell infusions as long as hematologic and other eligibility criteria have | | |
| | been met. | | |
| | ¹³¹I-MIBG therapy: Patients are eligible ≥6 weeks after therapeutic | | |
| | ¹³¹I-MIBG provided that all other eligibility criteria are met. | | |
| | Patients undergoing a major surgical procedure or laparoscopic | | |
| | procedure are eligible for enrollment after at least 28 days of the | | |
| | procedure, 14 days after an open biopsy. | | |
| | Central line placement or subcutaneous port placement is not | | |
| | considered major surgery. | | |
| | Core biopsy within 3 days prior to enrollment | | |
| | Fine needle aspirate within 3 days prior to enrollment | | |
| | Surgical or other wounds must be adequately healed prior to | | |
| | enrollment. | | |
| | 9. Have received at least one front line therapy for the treatment of their | | |
| | malignancy; on the chemotherapy combination arms, patients may have | | |
| | received prior treatment with the same agent. | | |
| | 10. Have adequate organ and marrow function on first day of study treatment as | | |
| | follows: | | |
| | a. For single agent 9-ING-41: ANC ≥500/mm³ | | |
| | For 9-ING-41 plus chemotherapy: ANC ≥750/mm³ | | |
| | b. For single agent 9-ING-41: Platelets ≥50,000/mm³ | | |
| | For 9-ING-41 plus chemotherapy: Platelets ≥75,000/mm³ | | |
| | c. Patients known to have bone marrow involvement with neuroblastoma | | |
| | are eligible provided that minimum ANC and platelet count criteria are | | |
| | met but are not evaluable for hematological toxicity. | | |
| | d. Total bilirubin ≤1.5 × ULN for age AND SGPT (ALT) ≤5.0 × ULN | | |
| | for age (≤225 U/L). For the purpose of this study, the ULN for SGPT | | |
| | is 45 U/L. | | |
| | e. Creatinine clearance or estimated radioisotope GFR ≥70 | | |
| | mL/min/1.73 m² or a serum creatinine based on age/gender as follows: | | |
| | | Maximum Serum | Maximum Serum |
| | | Creatinine | Creatinine |
| | | (mg/dL) | (mg/dL) |
| | Age | Male | Female |
| | 1 month to <6 months | 0.4 | 0.4 |
| | 6 months to <1 year | 0.5 | 0.5 |
| | 1 to <2 years | 0.6 | 0.6 |
| | 2 to <6 years | 0.8 | 0.8 |
| | 6 to <10 years | 1 | 1 |
| | 10 to <13 years | 1.2 | 1.2 |
| | 13 to <16 years | 1.5 | 1.4 |
| | ≥16 years | 1.7 | 1.4 |

| | 11. Pregnancy tests must be obtained in girls who are post-menarchal. Girls of | |
|---|---|---|
| | childbearing potential must have a negative baseline blood or urine | |
| | pregnancy test within 72 hours of first study therapy. Patients of | |
| | childbearing potential must agree to use hormonal or barrier birth control | |
| | with spermicidal gel, or total abstinence to avoid pregnancy for the duration | |
| | of study participation and in the following 100 days after discontinuation of | |
| | study treatment. | |
| | 12. All patients and/or their parents or legal guardians must sign a written | |
| | informed consent. The investigational nature and objectives of the trial, the | |
| | procedures and treatments involved and their attendant risks and | |
| | discomforts, and potential alternative therapies will be carefully explained to | |
| | the patient or the patient's parents or guardian if the patient is a child, and a | |
| | signed informed consent and assent will be obtained according to | |
| | institutional guidelines. | |
| Population | Exclusion Criteria: | |
| (cont'd) | Patients who meet any of the following criteria will be excluded from trial entry: | |
| | 1. Has hypersensitivity to any of the components of 9-ING-41 and/or | |
| | chemotherapy or to the excipients used in their formulation | |
| | 2. Has uncontrolled concurrent illness that would limit compliance with study | |
| | requirements | |
| | 3. Has clinically significant retinal disease | |
| | 4. Patients with symptoms of congestive heart failure are not eligible. | |
| | 5. Has current malignancy other than the target malignancy with the exception | |
| | of surgically treated local tumors or is currently receiving other anti-cancer | |
| | therapies, including radiation. | |
| | 6. Has not recovered from clinically significant toxicities as a result of prior | |
| | anticancer therapy, except alopecia, infertility and ototoxicity. Recovery is | |
| | defined as ≤Grade 2 severity per Common Terminology Criteria for | |
| | Adverse Events (CTCAE) Version 5.0 (v5.0). | |
| | 7. Is pregnant or lactating | |
| | 8. Has received a prior solid organ transplantation | |
| | 9. Is receiving any other investigational medicinal product or participating in | |
| | another interventional clinical trial | |
| | 10. For neuroblastoma-patients with only bone marrow detectable disease | |
| | (bone marrow aspirate or trephine) are NOT eligible for the study. | |
| Treatments & | 9-ING-41 as a Single Agent: | |
| Regimens | 9-ING-41 is administered by intravenous infusion twice weekly, on Days 1 | |
| | and 4 of each week, for cycle duration of 21 days. | |
| | Treatment is continued until disease progression, unacceptable toxicity, or | |
| | consent withdrawal. | |
| | The starting dose level on this study corresponds to 62% of the highest currently | |
| | tolerated dose level in the 1801 adult study (15 mg/kg). | |
| | Dose Levels: | |
| | Dose Level 3 | 15.0 mg/kg/dose |
| | Dose Level 2 | 12.4 mg/kg/dose |
| | Dose Level 1 | 9.3 mg/kg/dose |
| | (Starting Dose Level) | |
| | Dose Level −1 | 7.0 mg/kg/dose |

| 9-ING-41 and Irinotecan: | |
|---|---|
| 9-ING-41 was given at Dose Level 1, or 9.3 mg/kg/dose, and escalation | |
| followed a rolling 6 design as per single agent dose levels. Dose de-escalation of | |
| 9-ING-41 is permitted for patients experiencing suspected 9-ING-41-related | |
| toxicity. | |
| Irinotecan is given at the following dose and schedule: | |
| Irinotecan 50 mg/m²/day administered over 90 minutes IV on Days 1-5 | |
| every 21 days (cycle duration is 21 days). | |
| On days when administered with Irinotecan, 9-ING-41 infusion should | |
| begin no earlier than 1 hour after the end of irinotecan infusion. | |
| Irinotecan dose de-escalation is allowed for irinotecan-related toxicity as per | |
| following schedule below: | |
| Dose Level 1 | 50 mg/m²/day |
| (Starting dose level) | |
| Dose Level −1 | 40 mg/m²/day |
| Dose Level −2 | 30 mg/m²/day |
| Dose Level −3 | 20 mg/m²/day |

| 9-ING-41 plus Temozolomide/Irinotecan: | | | | | | | | |
|---|---|---|---|---|---|---|---|---|
| Treatment cycles is repeated every 21 days (3 weeks): | | | | | | | | |
| Day 1 | Day 2 | Day 3 | Day 4 | Day 5 | Day 8 | Day 11 | Day 15 | Day 18 |
| TMZ | TMZ | TMZ | TMZ | TMZ | | | | |
| IRIN | IRIN | IRIN | IRIN | IRIN | | | | |
| 9-ING- | | | 9-ING- | | 9-ING- | 9-ING- | 9-ING- | 9-ING- |
| 41 | | | 41 | | 41 | 41 | 41 | 41 |

| TMZ = Temozolomide 100 mg/m²/dose PO daily on Days 1-5; given at least | | | | | | | | |
|---|---|---|---|---|---|---|---|---|
| 1 hour prior to irinotecan. For patients whose body surface area (BSA) is | | | | | | | | |
| <0.5 m², temozolomide dosing is based on body weight in kg. For patients | | | | | | | | |
| with a BSA <0.5 m², 2.5 mg/kg is used. | | | | | | | | |
| IRIN = Irinotecan: 50 mg/m²/dose IV daily on Days 1-5 (90 min infusion). | | | | | | | | |
| 9-ING-41: Treatment starts at Dose Level 1, or 9.3 mg/kg/dose, and is | | | | | | | | |
| escalated as per single agent dose levels. Dose de-escalation of 9-ING-41 is | | | | | | | | |
| permitted for patients experiencing suspected 9-ING-41-related toxicity. | | | | | | | | |
| Treatment is given twice a week on Days 1 and 4 of every week, for cycle | | | | | | | | |
| length of 21 days. (Infusion expected to require 2-4 hours.) | | | | | | | | |
| On days when administered with irinotecan and temozolomide, 9-ING-41 | | | | | | | | |
| infusion should begin no earlier than 1 hour after the end of | | | | | | | | |
| irinotecan infusion and 2.5 hours from TMZ oral administration. | | | | | | | | |
| Diarrhea prophylaxis per institutional standards | | | | | | | | |
| Pneumocystis prophylaxis per institutional standards | | | | | | | | |
| 9-ING-41 plus Cyclophosphamide/Topotecan: | | | | | | | | |
| Treatment cycles are repeated every 21 days (3 weeks): | | | | | | | | |
| Day 1 | Day 2 | Day 3 | Day 4 | Day 5 | Day 9 | Day 12 | Day 16 | Day 19 |
| CYCLO | CYCLO | CYCLO | CYCLO | CYCLO | | | | |
| TOPO | TOPO | TOPO | TOPO | TOPO | | | | |
| | 9-ING- | | | 9-ING-41 | 9-ING- | 9-ING- | 9-ING- | 9-ING- |
| | 41 | | | | 41 | 41 | 41 | 41 |

| CYCLO = Cyclophosphamide. The dose of cyclophosphamide is fixed at | |
|---|---|
| 400 mg/m²/dose for BSA ≥0.6 m². For BSA <0.6 m² see dosing table below: | |
| CYCLOPHOSPHAMIDE | |
| BSA (m²) | Dose |
| 0.25-0.29 | 68 mg |
| 0.3-0.34 | 100 mg |
| 0.35-0.39 | 124 mg |
| 0.4-0.44 | 148 mg |
| 0.45-0.49 | 180 mg |
| 0.5-0.54 | 200 mg |
| 0.55-0.59 | 220 mg |
| ≥0.6 | 400 mg/m² |

| | Cyclophosphamide is administered intravenously over 30 min once daily for | | | | |
|---|---|---|---|---|---|
| | 5 days every 21 days. | | | | |
| | TOPO = Topotecan. The dose of topotecan is fixed at 1.2 mg/m²/dose for | | | | |
| | BSA ≥0.6 m². For BSA <0.6 m² see dosing table below: | | | | |
| | TOPOTECAN | | | | |
| | BSA (m²) | | Dose | | |
| | 0.25-0.29 | | 0.32 mg | | |
| | 0.3-0.34 | | 0.38 mg | | |
| | 0.35-0.39 | | 0.44 mg | | |
| | 0.4-0.44 | | 0.5 mg | | |
| | 0.45-0.49 | | 0.56 mg | | |
| | 0.5-0.54 | | 0.62 mg | | |
| | 0.55-0.59 | | 0.68 mg | | |
| | ≥0.6 | | 1.2 mg/m² | | |
| | Topotecan is administered, after cyclophosphamide, intravenously over 30 | | | | |
| | min once daily for 5 days every 21 days. | | | | |
| | 9-ING-41: Treatment starts at Dose Level 1, or 9.3 mg/kg/dose, and is | | | | |
| | escalated as per single agent dose levels. Dose de-escalation of 9-ING-41 is | | | | |
| | permitted for patients experiencing suspected 9-ING-41-related toxicity. | | | | |
| | Treatment is given twice a week on Days 2 and 5 of every week, for cycle | | | | |
| | duration of 21 days. (Infusion expected to require 2-4 hours.) | | | | |
| | On days when administered with cyclophosphamide and topotecan, | | | | |
| | 9-ING-41 infusion should begin no earlier than 1 hour after the end of | | | | |
| | topotecan infusion. | | | | |
| | Growth factor support should be administered per institutional standards. | | | | |
| Duration of | Patients may continue to receive subsequent cycles of therapy as long as they do | | | | |
| Therapy | not have clinically significant progressive disease and/or unacceptable toxicity | | | | |
| | or consent withdrawal. | | | | |
| Schedule of | 9-ING-41: Intravenous infusion on Days 1 and 4 of each week for a 3-week | | | | |
| Administration | (21 day) cycle when given as a single agent or in irinotecan-containing arms. | | | | |
| | For cyclophosphamide/topotecan arm, 9-ING-41 is given on Days 2 and 5 of | | | | |
| | every week in a 21-day cycle. | | | | |
| | Irinotecan: Intravenous infusion for 90 min given on Days 1-5 and repeated | | | | |
| | every 21 days, for a 21-day cycle. | | | | |
| | When given on the same day as 9-ING-41, end of infusion is at least 1 hour | | | | |
| | before start of 9-ING-41 infusion. | | | | |
| | In the temozolomide arm, administer at least 1 hour after temozolomide. | | | | |
| | Temozolomide: PO or via NG or G-tube, preferably on an empty stomach, at | | | | |
| | least 1 hour before or 2 hours after meals. Given on Days 1-5 of a 21-day cycle. | | | | |
| | Administer at least 1 hour prior to irinotecan administration. | | | | |
| | When given on the same day as 9-ING-41, end of administration is at least 2.5 | | | | |
| | hours before start of 9-ING-41 infusion. | | | | |
| | Cyclophosphamide: IV over 15-30 minutes, given on Days 1-5 of a 21-day | | | | |
| | cycle. | | | | |
| | Topotecan: IV over 30 min, given on Days 1-5 of a 21-day cycle. Administered | | | | |
| | after cyclophosphamide. | | | | |
| | When given on the same day as 9-ING-41, end of infusion is at least 1 hour | | | | |
| | before start of 9-ING-41 infusion. | | | | |
| Safety | Safety is assessed throughout the study including by recording and monitoring | | | | |
| Evaluation | Adverse events (AEs) (CTCAE v5.0), vital signs (blood pressure, pulse, | | | | |
| | respiratory rate, body temperature), physical examination findings, serum | | | | |
| | chemistry and hematology laboratory values, urinalysis, ECG, and concomitant | | | | |
| | medication usage. | | | | |
| | DLT is defined as any of the following events that are possibly, probably, or | | | | |
| | definitely attributable to 9-ING-41 and/or chemotherapy that occur within the | | | | |
| | first cycle of therapy (21 days). | | | | |
| | Dose-limiting hematological and non-hematological toxicities are defined | | | | |
| | differently. | | | | |
| | Non-Hematological Dose-Limiting Toxicity: | | | | |
| | Any clinically significant Grade 3 or Grade 4 non-hematological toxicity | | | | |
| | attributable to the 9-ING-41 with the specific exclusion of: | | | | |
| | Grade 3 liver enzyme elevation, including ALT/AST that returns to | | | | |
| | levels that meet eligibility criteria within 7 days of study drug | | | | |
| | interruption and that does not recur upon study drug re-challenge | | | | |
| | Grade 3 bilirubin that returns to levels that meet the eligibility criterion | | | | |
| | within 7 days of study drug interruption and that does not recur upon | | | | |
| | study drug re-challenge | | | | |
| | Grade 3 diarrhea ≤3 days duration | | | | |
| | Grade 3 nausea and vomiting of less <3 days duration | | | | |
| | Grade 3 or 4 fever <5 days duration | | | | |
| | Grade 3 infection <5 days duration | | | | |
| | Grade 3 hypophosphatemia, hypokalemia, hypocalcemia, or | | | | |
| | hypomagnesemia responsive to oral supplementation | | | | |
| | Grade 3 or 4 amylase or lipase elevation that is not associated with | | | | |
| | symptoms or clinical manifestations of pancreatitis | | | | |
| | Delay in more than 14 days in receiving the next scheduled cycles due to | | | | |
| | persistent treatment related toxicities. | | | | |
| | Hematological Dose-Limiting Toxicity: | | | | |
| | Grade 4 neutropenia of >7 days duration | | | | |
| | Grade ≥4 thrombocytopenia | | | | |
| | Grade 3 thrombocytopenia of ≥7 days duration or is associated with | | | | |
| | clinically significant bleeding | | | | |
| Efficacy | Response and progression are evaluated using the revised Response Evaluation | | | | |
| Evaluation | Criteria in Solid Tumors (RECIST) guideline (version 1.1) or specific standard | | | | |
| | response criteria appropriate to the patient's malignancy, including the Revised | | | | |
| | International Neuroblastoma Response Criteria (INRC) for neuroblastoma. | | | | |
| | Evaluable for objective response: Patients who exhibit objective disease | | | | |
| | progression prior to the end of cycle 1 are considered evaluable for response. | | | | |
| | For all other patients, only those patients who have measurable disease present | | | | |
| | at baseline, have received at least one cycle of therapy, and have had their | | | | |
| | disease re-evaluated are considered evaluable for response. Patients with | | | | |
| | neuroblastoma who have MIBG positive lesions that do not meet the definitions | | | | |
| | of measurable disease are considered evaluable for response according to MIBG | | | | |
| | response. | | | | |
| | Evaluable non-target disease response: Patients who have lesions present at | | | | |
| | baseline that are evaluable but do not meet the definitions of measurable | | | | |
| | disease, have received at least one cycle of therapy, and have had their disease | | | | |
| | re-evaluated are considered evaluable for non-target disease. The response | | | | |
| | assessment is based on the presence, absence, or unequivocal progression of the | | | | |
| | lesions. | | | | |
| | Measurable disease: Measurable lesions are defined as those that can be | | | | |
| | accurately measured in at least one dimension (longest diameter to be recorded) | | | | |
| | as ≥20 mm by chest x-ray, as ≥10 mm with CT scan, or ≥10 mm with calipers | | | | |
| | by clinical exam. All tumor measurements must be recorded in millimeters | | | | |
| | (or decimal fractions of centimeters). | | | | |
| | Non-measurable disease: All other lesions (or sites of disease), including small | | | | |
| | lesions (longest diameter <10 mm or pathological lymph nodes with ≥10 to <15 | | | | |
| | mm short axis), are considered non- measurable disease. Bone lesions, | | | | |
| | leptomeningeal disease, ascites, isolated bone marrow disease, germ cell or | | | | |
| | other tumor markers, pleural/pericardial effusions, lymphangitis | | | | |
| | cutis/pneumonitis, inflammatory breast disease, PET positive lesions and | | | | |
| | abdominal masses (not followed by CT or MRI), are considered as non- | | | | |
| | measurable. | | | | |
| | Neuroblastoma-specific response criteria: Revised International Neuroblastoma | | | | |
| | Response Criteria (INRC) for disease assessment. The updated response criteria | | | | |
| | incorporate current approaches to imaging of neuroblastoma, including | | | | |
| | functional imaging. Furthermore, a standardized approach to assessment of bone | | | | |
| | marrow involvement is included. The current INRC do not include methods of | | | | |
| | disease assessment that are less sensitive and/or specific for neuroblastoma (⁹⁹Tc | | | | |
| | bone scan and catecholamine levels). | | | | |
| Statistical | Phase 1: | | | | |
| Considerations- | The rolling six phase 1 trial design is being used for the conduct of the Phase 1 | | | | |
| Methods | portion of the study. Two to 6 patients can be concurrently enrolled onto a dose level, | | | | |
| Summary | dependent upon (1) the number of patients enrolled at the current dose level, (2) the | | | | |
| | number of patients who have experienced DLT at the current dose level, and (3) the | | | | |
| | number of patients entered but with tolerability data pending at the current dose level. | | | | |
| | Accrual is suspended when a cohort of six has enrolled or when the study endpoints | | | | |
| | have been met. | | | | |
| | Enrollment of patients began at the starting dose level in the single | | | | |
| | agent 9-ING-41 arm of the study following the rolling six trial design | | | | |
| | and continues until a RP2D has been reached for 9-ING-41 | | | | |
| | monotherapy. | | | | |
| | Enrollment on the irinotecan combination arm began at the starting | | | | |
| | single agent 9 ING-41 dose level once that was deemed safe and | | | | |
| | followed a rolling six design for a total of 3 dose levels. | | | | |
| | Enrollment on the temozolomide/irinotecan combination will begin | | | | |
| | after institutional protocol approval and follow a rolling six design for a | | | | |
| | total of 3 dose levels. | | | | |
| | Enrollment on the cyclophosphamide/topotecan combination arm will | | | | |
| | begin after institutional protocol approval and follow a rolling six | | | | |
| | design for a total of 3 dose levels. | | | | |
| | Sample Size: It is anticipated that 9-18 patients will be enrolled in each of the | | | | |
| | four treatment regimens for a total of 36-72 patients in Phase 1. | | | | |
| | Phase 2: | | | | |
| | After completion of enrollment on the Phase 1 portion of the study, and | | | | |
| | determination of a RP2D for these regimens, a neuroblastoma-specific cohort | | | | |
| | opens. This is a signal-seeking Phase 2 cohort for refractory neuroblastoma | | | | |
| | patients. | | | | |
| | The primary objective is to assess the ORR (CR + PR) of the 9-ING-41-based | | | | |
| | combination regimens (cyclophosphamide/topotecan and | | | | |
| | temozolomide/irinotecan) via INRC criteria and pick a regimen to move forward | | | | |
| | in confirmatory studies. Secondary objectives include the assessment of other | | | | |
| | efficacy variables, including PFS, duration of tumor response, 1-year survival | | | | |
| | rate and OS, as well as additional evaluation of toxicities. | | | | |
| | Categorical endpoints (ORR) are summarized with frequencies, percentages, | | | | |
| | and 2-sided 95% exact confidence intervals. Time-to event endpoints (DoR, | | | | |
| | PFS, and OS) are summarized by Kaplan-Meier methods including median, | | | | |
| | 95% CI, number of events, number censored and Kaplan-Meier figures. | | | | |
| | Sample Size: The minimum number of responders needed to continue to the | | | | |
| | next stage is determined based on the Simon's 2-stage design, with 80% power | | | | |
| | and 2-sided significance level of 0.05 (Type 1 error rate). An ORR of 20% is | | | | |
| | hypothesized (alternative hypothesis). An ORR of 5% (null hypothesis) is | | | | |
| | considered the lower threshold activity based on historical data in the refractory | | | | |
| | neuroblastoma population. | | | | |
| | Both regimens (9-ING-41 + either temozolomide/irinotecan or | | | | |
| | cyclophosphamide/topotecan) enroll into Stage 1 of the Simon 2-stage design | | | | |
| | (n = 10 per cohort, n = 20 total). After Stage 1 enrollment is complete | | | | |
| | (neuroblastoma patients enrolled in the safety lead-in arms count toward total n | | | | |
| | of each arm in Stage 1), an interim analysis is performed to determine which | | | | |
| | regimen goes forward into Stage 2 (n = 19). At the completion of the study, of the | | | | |
| | 39 patients (20 at Stage 1, 19 at Stage 2), 29 will have received the Stage 2 | | | | |
| | regimen. If ≥4 of these 29 patients have CR or PR, further evaluation may be | | | | |
| | pursued with confirmatory studies. | | | | |
| | | | | | Stage 2 |
| | | Stage 1 | Stage 1 | Interim | success |
| | Regimen | futility | continue | Analysis | criteria |
| | 9-ING-41 + | 0/10 | ≥1/10 | | |
| | cyclophosphamide/ | (probability | | | |
| | topotecan | 60%) | | | |
| | | | | Intermim | |
| | | | | Analysis | |
| | | | | Pick one | ≥4/29 |
| | | | | regimen | |
| | | | | For Stage 2 | |
| | 9-ING-41 + | 0/10 | ≥1/10 | | |
| | temozolomide/ | (probability | | | |
| | irinotecan | 60%) | | | |

It is planned that 39 evaluable patients will be enrolled in Phase 2.

### Results:

9-ING-41 was well tolerated in this heavily pre-treated pediatric patient population. The infusion volume limitation and twice per week dosing schedule highlight the need for oral dosage forms.

One patient with recurrent Ewing's sarcoma had a radiographic and pathologic complete response after 3 cycles of elraglusib/cyclophosphamide/topotecan. 6 patients (26.1%) had stable disease (2 NBL, 1 aRMS, 1 EP, 1 OS, 1 GBM). 8 pts (35%) remained on study treatment ≥3 months (2 NBL, 2 EP, 1 OS, 1 aRMS, 1 ES, 1 PB). Median treatment duration was 40 days (range 1-126).

TABLE 1
Intermediate Data

| | | 9-ING- | | OS | |
|---|---|---|---|---|---|
| Tumor Type | Tumor Group | 41 Dose | Combo Treatment | (days) | Notes |
| Alveolar | Rhabdomyo- | 9.3 | | | |
| rhabdomy- | sarcoma | mg/kg | Irinotecan | 375 | |
| sarcoma | | | | | |
| Embryonal CNS | Embryonal | 9.3 | Irinotecan | 262 | |
| tumor NOS | CNS | mg/kg | | | |
| Neuroblastoma | Neuroblastoma | 9.3 | Irinotecan | 245 | |
| | | mg/kg | | | |
| Osteosarcoma | Osteosarcoma | 9.3 | Irinotecan | 234 | |
| | | mg/kg | | | |
| Osteosarcoma | Osteosarcoma | 9.3 | Irinotecan | 108 | |
| | | mg/kg | | | |
| Neuroblastoma | Neuroblastoma | 9.3 | Irinotecan | 389 | |
| | | mg/kg | | | |
| Neuroblastoma | Neuroblastoma | 12.4 | Irinotecan | 360 | |
| | | mg/kg | | | |
| Neuroblastoma | Neuroblastoma | 12.4 | Irinotecan | 76 | |
| | | mg/kg | | | |
| Progressive | Ependymoma | 9.3 | Cyclophosphamide/ | 407 | |
| ependymoma | | mg/kg | topotecan | | |
| Ewing sarcoma | Ewing Sarcoma | 9.3 | Cyclophosphamide/ | 379 | pCR- |
| | | mg/kg | topotecan | | complete |
| | | | | | response |
| Osteosarcoma | Osteosarcoma | 9.3 | Cyclophosphamide/ | 389 | |
| | | mg/kg | topotecan | | |
| Rhabdomyo- | Rhabdomyo- | 15 | Irinotecan | 19 | |
| sarcoma | sarcoma | mg/kg | | | |
| Glioblastoma | Glioma | 12.4 | Cyclophosphamide/ | 406 | |
| multiforme | | mg/kg | topotecan | | |
| (GBM) | | | | | |
| Recurrent | Ependymoma | 15 | Irinotecan | 403 | |
| ependymoma | | mg/kg | | | |
| Neuroblastoma | Neuroblastoma | 12.4 | Cyclophosphamide/ | 308 | pCR- |
| | | mg/kg | topotecan | | complete |
| | | | | | response |
| Ewing sarcoma | Ewing Sarcoma | 12.4 | Cyclophosphamide/ | 176 | |
| | | mg/kg | topotecan | | |
| CIC rearranged | Ewing Sarcoma | 15 | Cyclophosphamide/ | 47 | |
| sarcoma | | mg/kg | topotecan | | |
| Rhabdomyo- | Rhabdomyo- | 15 | Cyclophosphamide/ | 78 | |
| sarcoma | sarcoma | mg/kg | topotecan | | |
| Neuroblastoma | Neuroblastoma | 15 | Irinotecan | 68 | |
| | | mg/kg | | | |
| High grade | Astrocytoma | 15 | N/A | 97 | |
| astrocytoma | | mg/kg | | | |
| Adrenocortical | Adrenocortical | 15 | Cyclophosphamide/ | 102 | |
| carcinoma | | mg/kg | topotecan | | |
| Neuroblastoma | Neuroblastoma | 15 | Cyclophosphamide/ | 113 | |
| | | mg/kg | topotecan | | |

“OS” = overall survival;
“CR”—complete response

The 5 years survival rate for newly diagnosed Ewing sarcoma patients is >70% but patients that have recurrent (relapsed) disease, like the patients enrolled in this study, have 5 years survival <30%. Patients that have metastasis and disease progression despite 2 or more chemotherapy regimens have very short survival <6 months. There are no treatment regimens that meaningfully extend life in Ewing sarcoma patients with metastatic, refractory disease.

Clinical data has been obtained in patients with refractory pediatric malignancies treated with the combination of 9-ING-41 and cyclophosphamide/topotecan. The patients that were enrolled in this study were patients that had previously been treated with standard of care treatments in their particular disease and had disease progression despite these treatments. Of 25 evaluable patients enrolled in this study, seven (7) were enrolled with metastatic, refractory Ewing and Ewing-like sarcoma.

The seven Ewing patients enrolled in this trial all appear to have metastatic disease and had disease progression prior to joining the study despite previous chemotherapy and radiation. Four of the seven patients had received two or more previous chemotherapy regimens. All patients received the combination of elraglusib+cyclophosphamide/topotecan. One patient had complete remission of their tumor at their 1st tumor assessment (2 months of treatment), stopped all treatments after 4 months and continues to be in remission with no evidence of disease almost 2 years after termination of treatment. A second patient had a partial remission with 52% reduction of tumor at the 1st tumor assessment; and a third patient has a partial remission with 96% reduction in tumor at the 1st tumor assessment. Four of the seven patients remain alive and three of the seven continue on treatment. See Table 2.

TABLE 2
Survival and best response for Ewing's sarcoma patients treated with
elraglusib and chemotherapy in this trial (unaudited data)

| | | | Status | | |
|---|---|---|---|---|---|
| Tumor | Elraglusib | Chemotherapy | Last | Survival | Best |
| Type | Dose | Backbone | FUP | (days) | Response |
| Ewing | 9.3 mg/kg | Cyclophosphamide/ | Alive | 804 | CR |
| sarcoma | | topotecan | | | |
| Ewing | 12.4 mg/kg | Cyclophosphamide/ | Dead | 177 | SD |
| sarcoma | | topotecan | | | |
| CIC | 15 mg/kg | Cyclophosphamide/ | Dead | 48 | PD |
| rearranged | | topotecan | | | |
| sarcoma | | | | | |
| Ewing | 15 mg/kg | Cyclophosphamide/ | Dead | 272 | SD |
| sarcoma | | topotecan | | | |
| Ewing | 15 mg/kg | Cyclophosphamide/ | Alive | 318 | NAP |
| sarcoma | | topotecan | | | |
| Ewing | 15 mg/kg | Cyclophosphamide/ | Alive | 145 | PR |
| sarcoma | | topotecan | | | |
| Ewing | 15 mg/kg | Cyclophosphamide/ | Alive | 130 | PR |
| sarcoma | | topotecan | | | |

“FUP” = follow-up;
“CR” = complete response;
“PR” = partial response;
“PD” = progressive disease;
“SD” = stable disease;
“NAP” = no assessment possible.

TABLE 3
Survival and best response for other pediatric cancer patients treated
with elraglusib and chemotherapy in this trial (unaudited data)

| | | | Status | | |
|---|---|---|---|---|---|
| | Elraglusib | Chemotherapy | Last | Survival | Best |
| Tumor Type | Dose | Backbone | FUP | (days) | Response |
| Glioblastoma | 12.4 mg/kg | Cyclophosphamide/ | Alive | 407 | SD |
| multiforme | | topotecan | | | |
| (GBM) | | | | | |
| Neuroblastoma | 9.3 mg/kg | Irinotecan | Dead | 245 | SD |
| Neuroblastoma | 9.3 mg/kg | Irinotecan | Dead | 389 | PD |
| Neuroblastoma | 12.4 mg/kg | Irinotecan | Dead | 360 | SD |
| Neuroblastoma | 12.4 mg/kg | Cyclophosphamide/ | Dead | 438 | CR |
| | | topotecan | | | |
| Neuroblastoma | 15 mg/kg | Cyclophosphamide/ | Alive | 440 | SD |
| | | topotecan | | | |
| Rhabdomyo- | 9.3 mg/kg | Irinotecan | Dead | 375 | SD |
| sarcoma | | | | | |
| Osteosarcoma | 9.3 mg/kg | Cyclophosphamide/ | Alive | 481 | SD |
| | | topotecan | | | |
| Ependymoma | 12.4 mg/kg | N/A | Dead | 440 | SD |
| Ependymoma | 9.3 mg/kg | Cyclophosphamide/ | Alive | 182 | PD |
| | | topotecan | | | |
| Ependymoma | 15 mg/kg | Irinotecan | Dead | 757 | SD |
| Ependymoma | 15 mg/kg | Irinotecan | Dead | 526 | SD |

“FUP” = follow-up;
“CR” = complete response;
“PR” = partial response;
“PD” = progressive disease;
“SD” = stable disease.

### Example 3. Immunostimulatory Effects Towards NK and T-Cells by 9-ING-41 in Human Liposarcoma and Osteosarcoma Cell Lines

Saos-2 osteosarcoma and 93T449 liposarcoma cell lines were pretreated at IC50₇₂ with small molecule GSK-3 inhibitor 9-ING-41 (elraglusib). The cells were harvested for western blot analysis after 48 hours of treatment. Additionally, both cancer cell lines as well as TALL-104 T-cells and NK-92 NK cells were treated with 0.5 μM 9-ING-41 for 24 hours and harvested for Luminex cytokine profiling. The western blots demonstrated an increase in cPARP, an apoptotic marker, in both cancer cell lines after 9-ING-41 treatment. Additionally, an increase in PD-L1 expression was observed. The cytokine analysis revealed stimulation of immune cell activity in response to 9-ING-41. Treated T-cells had an increase in CXCL11, which is associated with T-cell recruitment, as well as an increased level of IL-18, which is shown to induce increased IFN-γ in Th1 cells. Additionally, NK-92 cells demonstrated an increase in IL-8 chemokine and an increase in soluble TRAIL (TRAIL/TNFSF10). The cancer cell lines showed a homogenous increase in growth factor TGF-α, however, only the Saos-2 osteosarcoma cell line demonstrated an increase of IL-6. Follow-up cytokine profiling is in progress. The increase in immunostimulatory cytokines as well as the increased expression of PD-L1 suggest a rationale for combining 9-ING-41 with immune checkpoint blockade therapy. The potential synergistic effect of these two therapies is currently under investigation with co-culture experiments of sarcoma and immune cells. The treatment cohorts for these experiments include 9-ING-41 combined with either anti-PD-L1, anti-PD-1, or anti-CTLA-4 immune checkpoint inhibitors. These results suggest a promising combination therapy strategy for patients with soft tissue and bone sarcomas and future work will strive to better elucidate the mechanisms of efficacy.

1. A method of treating cancer in a pediatric patient in need thereof, comprising administering to the pediatric patient 9-ING-41 in combination with an additional therapeutic agent.

2. The method of claim 1, wherein the cancer is an alveolar rhabdomyosarcoma, embryonal CNS tumor NOS, neuroblastoma, osteosarcoma, progressive ependymoma, Ewing's sarcoma, rhabdomyosarcoma, pediatric glioblastoma multiforme (GBM), diffuse interstitial pontine glioma (DIPG)/diffuse midline glioma (DMG), Wilm's tumor, recurrent ependymoma, CIC rearranged sarcoma, high grade astrocytoma, adrenocortical carcinoma, embryonal CNS, ependymoma, glioma, astrocytoma, or adrenocortical.

3. The method of claim 1, wherein the cancer is a sarcoma.

4. The method of claim 3, wherein the sarcoma is an alveolar rhabdomyosarcoma, osteosarcoma, Ewing sarcoma, rhabdomyosarcoma, or CIC rearranged sarcoma.

5. The method of claim 4, wherein the sarcoma is alveolar rhabdomyosarcoma.

6. The method of claim 4, wherein the sarcoma is rhabdomyosarcoma.

7. The method of claim 4, wherein the sarcoma is osteosarcoma.

8. The method of claim 4, wherein the sarcoma is Ewing sarcoma.

9. The method of claim 4, wherein the sarcoma is CIC rearranged sarcoma.

10. The method of claim 1, wherein the cancer is a refractory or recurrent cancer.

11. The method of claim 1, wherein the 9-ING-41 is administered in an amount of about 5-20 mg/kg of patient weight.

12. The method of claim 11, wherein the 9-ING-41 is administered at a frequency of twice per week over a 21-day cycle.

13. The method of claim 11, wherein the 9-ING-41 is administered in an amount of about 9.3 mg/kg administered at a frequency of twice per week over a 21-day cycle.

14. The method of claim 11, wherein the 9-ING-41 is administered in an amount of about 12.4 mg/kg administered at a frequency of twice per week over a 21-day cycle.

15. The method of claim 11, wherein the 9-ING-41 is administered in an amount of about 15 mg/kg administered at a frequency of twice per week over a 21-day cycle.

16. The method of claim 1, wherein the additional therapeutic agent comprises irinotecan, cyclophosphamide, topotecan, temozolomide, a PI3 kinase inhibitor, a protein kinase B (AKT) inhibitor, a mTOR inhibitor, PD-L1 inhibitor, a PD-1 inhibitor, or a CTLA-4 inhibitor.

17. The method of claim 16, wherein the additional therapeutic agent comprises irinotecan.

18. The method of claim 17, wherein the irinotecan is administered intravenously over 90 minutes in an amount of 50 mg/m2/day on days 1-5 of every 21 day cycle.

19. The method of claim 16, wherein the additional therapeutic agent comprises temozolomide.

20. The method of claim 19, wherein the temozolomide is administered orally in an amount of 100 mg/m2/dose on days 1-5 of every 21 day cycle.

21. The method of claim 16, wherein the additional therapeutic agent comprises cyclophosphamide.

22. The method of claim 21, wherein the cyclophosphamide is administered intravenously over 30 minutes in an amount of 400 mg/m2/dose on days 1-5 of every 21 day cycle.

23. The method of claim 16, wherein the additional therapeutic agent comprises topotecan.

24. The method of claim 23, wherein the topotecan is administered intravenously over 30 minutes in an amount of 1.2 mg/m2/dose once on Days 1 through 5.

25. The method of claim 16, wherein the additional therapeutic agent comprises a PI3 kinase inhibitor.

26. The method of claim 16, wherein the additional therapeutic agent comprises a mTor inhibitor.

27. The method of claim 1, wherein the patient is 0-23 years old.

28. The method of claim 27, wherein the patient is 0-18 years old.

29. The method of claim 27, wherein the patient is 0-14 years old.
